(19) United States
(12) Patent Application Publication
(10) Pub. No.: US 2026/0157755 A1
(43) Pub. Date: Jun. 11, 2026

(54) METAL-CONTAINING SURGICAL ADJUNCTS FOR TUNABLE ABSORPTION RATE

(71) Applicant: CILAG GMBH INTERNATIONAL, Zug (CH)

(72) Inventors: PEYTON HOPSON, Cincinnati, OH (US); Madelaine Franzoni, Cincinnati, OH (US)

(73) Assignee: CILAG GMBH INTERNATIONAL, Zug (CH)

(21) Appl. No.: 19/412,063

(22) Filed: Dec. 8, 2025

Related U.S. Application Data
(60) Provisional application No. 63/729,590, filed on Dec. 9, 2024.

(51) Int. Cl. A61B 17/072; A61B 17/00

(52) U.S. Cl. CPC A61B 17/07207; A61B 2017/00004; A61B 2017/00526; A61B 2017/07271

(57) ABSTRACT

The disclosed technology includes a metal-containing surgical adjunct for tunable absorption rate. The surgical adjunct may comprise a polyurethane (PU) foam and a metallic component or comprise a metal-containing PU foam wherein one or more metallic elements are incorporated into a backbone or a sidechain of polyurethane. The metal accelerates the degradation of the foam in vivo.

### CROSS-REFERENCE TO RELATED APPLICATION

This application claims benefit to U.S. Provisional Application Ser. No. 63/729,590, filed on December 9, 2024, which is hereby incorporated by reference in its entirety.

### FIELD

The present invention relates to surgical adjuncts comprising a polyurethane foam and a metallic component or comprising a metal-containing polyurethane foam.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and are often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

Some surgical cartridges may include an implantable adjunct comprising a buttress material that is supported on the deck of the cartridge or on one of the jaws. This implantable adjunct reinforces the staple line applied to the tissue and can be used to compensate for differences in tissue thickness. For instance, tissue may be thicker at one end of a staple cartridge than at another, yet the length of staple legs is the same for all staples in the cartridge. As such, a staple can have proper length in one section of that tissue yet be too long for another section of the tissue. An endoscopic stapling adjunct with an ideal amount of compression is strong enough to create and maintain a hemostatic seal, while also not over-compressing the tissue or too difficult to complete the firing. In some implementations, the implantable adjunct biodegrades over time.

The material choice of the adjunct is critical as it affects the adjunct absorption byproducts, the absorption time, the mode of adsorption (i.e., bulk vs. surface eroding), and the mechanical strength profile of the adjunct over the degradation.

A specific example of a stapling adjunct comprises a polyurethane (PU) foam. The primary absorption mechanism for aliphatic PU is oxidative and/or enzymatic. The correlation between the mechanical strength and the absorption rate of the adjunct is negative in nature (i.e. when one increases, the other decreases). There is a limitation in balancing the time an adjunct holds its mechanical strength during healing and the time an adjunct absorbs into the tissue in vivo. Further, there are limitations in the adjunct materials to accelerate the adjunct absorption. As such, there is an unmet need to address these limitations.

### SUMMARY

There is provided a surgical adjunct having a polyurethane (PU) foam and a metallic component, wherein the metallic component accelerates the degradation of the foam in vivo.

There is provided a method of preparing the surgical adjunct by adding a metallic component during a foaming process of the polyurethane; or depositing the metallic component onto the surface of the polyurethane foam by spray coating, dip coating, electrospray, pad printing, screen printing, material jetting, or direct deposition.

There is provided a surgical adjunct with a metal-containing polyurethane foam, wherein one or more metallic elements are incorporated into a backbone or a sidechain of polyurethane, and wherein the metallic element accelerates the degradation of the foam in vivo.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument.

FIG. 2A is a top view of a staple cartridge for use with the surgical stapling and severing instrument of FIG. 1.

FIG. 2B is a side view of the staple cartridge of FIG. 2A.

FIG. 3 is a side view of a staple in an unfired (pre-deployed) configuration that can be disposed within the staple cartridge of the surgical cartridge assembly of FIG. 2A.

FIG. 4 is a perspective view of a knife and firing bar (“E-beam”) of the surgical stapling and severing instrument of FIG. 1.

FIG. 5 is a perspective view of a wedge sled of a staple cartridge of the surgical stapling and severing instrument of FIG. 1.

FIG. 6A is a longitudinal cross-sectional view of an exemplary surgical cartridge assembly having a compressible non-fibrous adjunct attached to a top or deck surface of a staple cartridge.

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct.

FIG. 7 is a partial-schematic illustrating the adjunct of FIGS. 6A-6B in a tissue deployed condition.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 10%” may refer to the range of values from 9.0% to 11.0%.

In addition, throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “substantially free” of something, or “substantially pure”, and like characterizations, can include both being “at least substantially free” of something, or “at least substantially pure”, and being “completely free” of something, or “completely pure.” As used herein, the term “substantially” or “substantially comprised of” means at least 90% by volume.

As used herein, the term “comprising”, “containing” or “including” means that at least the named compound, element, particle, or method step is present in the composition, article or method, but does not exclude the presence of other compounds, materials, particles, method steps, even if the other such compounds, material, particles, method steps have the same function as what is named.

The term “adjunct” or “surgical adjunct” refers to a material that is used in conjunction with a surgical staple or surgical stapler. The “adjunct” and “surgical adjunct” are used interchangeably. The “adjunct” or “surgical adjunct” may comprise a buttress or a cushion. The “adjunct” or “surgical adjunct” may comprise additionally comprise a wide range of items such as coatings to tune the degradable profile, hemostatic agents to control bleeding, tissue adhesives and sealants, or surgical meshes for tissue support. In some embodiments, an “adjunct” or a “surgical adjunct” comprises a buttress and/or a cushion.

As used herein, “buttress” or “surgical buttress” refers to a material used to provide structural support during surgical procedures. It may be employed to reinforce tissues, support sutures or surgical staple, or stabilize anatomical structures, particularly in areas where the tissue may be weak or under significant stress.

As used herein, “cushion” or “surgical cushion” refers to a material used during surgical procedures to provide padding, support, and protection to tissues and organs. It helps to prevent damage from pressure, friction, or other mechanical forces that may occur during surgery. Surgical cushions may be made from different materials, including foam, gel, or other specialized substances designed to provide adequate cushioning and support. In some embodiments, the buttress comprises a cushion. In some embodiments, when buttress comprises a padding, “buttress” and “cushion” are used interchangeably.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. Suitable aliphatic groups include, but are not limited to, linear or branched, substituted or unsubstituted alkyl, alkenyl, alkynyl groups and hybrids thereof such as cycloalkyl, (cycloalkyl)alkyl, (cycloalkenyl)alkyl or (cycloalkyl)alkenyl.

The term “alkyl,” unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. In some embodiments, “cycloalkyl” (or “carbocycle”) refers to a monocyclic C₃-C₆ hydrocarbon that is completely saturated or that contains one or more units of unsaturation, but which is not aromatic, that has a single point of attachment to the rest of the molecule. A carbocycle can be, under certain circumstances, a bridged bicyclic or a fused ring such as, e.g., an ortho-fused carbocycle, a spirofused carbocycle, etc. Examples of “alkyl” groups include, but are not limited to, methyl, ethyl, propyl, isopropyl, butyl, iso-butyl, sec-butyl, tert- butyl, pentyl, hexyl, heptyl, cyclopropyl, cyclobutyl, cyclopentyl, cyclohexyl, cycloheptyl, norbornyl, and the like.

The term “alkylene” refers to a bivalent alkyl group. An “alkylene chain” is a polymethylene group, i.e., –(CH₂)ₙ–, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkoxy” means an alkyl radical attached through an oxygen linking atom, represented by -O-alkyl. For example, “(C₁-C₄)alkoxy” includes methoxy, ethoxy, propoxy, and butoxy.

The term “aryl,” used alone or as part of a larger moiety, refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of compounds described herein, “aryl” refers to an aromatic ring system which includes, but is not limited to, phenyl, naphthyl, anthracyl and the like, which may bear one or more substituents. It will be appreciated that an “aryl” group can comprise carbon and heteroatom ring members.

As described herein, compounds described herein may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. As used herein, “suitable substituent”, “substituent”, “optional substituent”, or the substituent for any optionally substituted group means a group that does not nullify the synthetic or pharmaceutical utility of the compounds described herein or the intermediates useful for preparing them. Examples of “suitable substituent”, “substituent”, “optional substituent”, or the substituent for any optionally substituted group include, but are not limited to: C₁-C₆alkyl, C₁-C₆alkenyl, C₁-C₆alkynyl, C₁-C₆alkoxy, phenyl, C₃-C₅heteroaryl, C₃-C₁₀cycloalkyl, C₃-C₁₀heterocycloalkyl, C₅-C₆aryloxy, -CN, -OH, oxo, halo, haloalkyl, -NO₂, −CO₂H, −NH₂, −NH(C₁−C₈alkyl), −N(C₁−C₈alkyl)₂, −NH(phenyl), −N(phenyl)₂, −CHO, −CO(C₁−C₆alkyl), −CO(phenyl), −CO₂(C₁−C₆alkyl), and −CO₂(phenyl). One of skill in the art can readily choose a suitable substituent based on the stability and pharmacological and synthetic activity of the compounds described herein. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.−

As used herein, “degradation” refers to a process of breaking down of a polymer chain for absorption as atoms, molecules, or ions into the surroundings (e.g., tissue, cells, fluid, and the like). Degradation includes breaking primary and secondary bonds via thermal degradation, photodegradation, catalytic or enzymatic degradation, oxidative degradation, ion degradation, and biodegradation.

The term “isocyanate” refers to an organic compound characterized by the functional group –N=C=O. The term “diisocyanate” refers to an isocyanate that contains two isocyanate groups (–N=C=O).

The term “polymerizable compound” means a compound containing one or more polymerizable groups. The term encompasses, for instance, monomers, macromers, oligomers, prepolymers, cross-linkers, and the like.

As used herein, “polymerizable groups” are groups that can undergo chain growth polymerization, such as free radical and/or cationic polymerization, for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyl lactam, N-vinylamide, polyester polyols, poloxamers, and styryl functional groups, and mixtures of any of the foregoing. Preferably, the free radical polymerizable groups include (meth)acrylates, (meth)acrylamides, polyester polyols, poloxamers, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

The term “polyol” refers to a compound containing multiple hydroxyl (–OH) groups. The term “diol” refers to a compound containing two hydroxyl (–OH) groups.

The term “polyurethane,” as used herein, refers to a polymeric reaction product of an isocyanate and a polyol, and is not limited to those polymers which include only urethane or polyurethane linkages. It is well understood by those of ordinary skill in the art of preparing polyurethanes that the polyurethane polymers may also include linkages such as allophanate, carbodiimide, and other linkages described herein in addition to urethane linkages.

The term “polydrug,” as used herein, refers to a single drug and/or a drug including multiple active pharmaceutical ingredients (APIs) in a single dosage form or implant.

The expressions “reaction system,” “reactive formulation,” “reaction product,” and “reactive mixture” are interchangeably used herein, and all refer to a combination of reactive compounds used to make the bioabsorbable material according to the disclosure.

The term “room temperature” refers to temperatures of about 20°C, this means referring to temperatures in the range 18° C to 25° C. Such temperatures will include 18° C, 19° C, 20° C, 21° C, 22° C, 23° C, 24° C and 25° C.

Unless otherwise expressed, the “weight percentage” (indicated as % wt. or wt. %) of a component in a composition refers to the weight of the component over the total weight of the composition in which it is present and is expressed as a percentage.

“Glass transition temperature” and “T_{g}” as referred to herein refers to the temperature at which a reversible transition from a hard glass condition into a rubber-elastic condition occurs.

The terms “NCO value” or “isocyanate value” as referred to herein is the weight percentage of reactive isocyanate (NCO) groups in an isocyanate, modified isocyanate, or isocyanate prepolymer compound.

As used herein, the term “metal” refers to a metal or a metal alloy. In some embodiments, the term “metal” refers to a metal that is an alkali metal, an alkali metal, an alkaline-earth metal, or a transition metal, or a metal alloy comprising at least two elements selected from an alkali metal, an alkali metal, an alkaline-earth metal, or a transition metal. In some embodiments, the term “metal” refers to a metal in its free form, a metal ion, or a covalently bonded metal. In some embodiments, the terms “metal”, “metallic atom”, and “metallic element” are used interchangeably.

As used herein, the term “metallic group” or “metallic moiety” refers to a metallic ion or a covalently bonded metal in a metallic compound.

As used herein, the term “metallic compound” refers to a compound comprising two or more metals, or a compound comprising one or more metallic elements and one or more non-metallic elements.

As used herein, the term “metal-containing polyurethane” or “metal-containing PU” refers to a polyurethane polymer in which there is a chemical bond between a metal and a part of the polyurethane. The chemical bond may be an ionic bond or a covalent bond.

As used herein, the term “organometallic compound” refers to a compound containing at least one chemical bond between a carbon atom of an organic molecule and a metal.

As used herein, the term “metal-organic framework” or “MOF” refers to a compound containing metal ions or clusters coordinated to organic molecules. In some embodiments, the MOF contains metal ions or clusters coordinated to organic molecules comprising PU.

Surgical stapling assemblies and methods for manufacturing and using the same are provided. In general, a surgical stapling assembly can include a staple cartridge having staples disposed therein and an adjunct configured to be releasably retained on the staple cartridge. As discussed herein, the various adjuncts provided can be configured to compensate for variations in tissue properties, such as variations in tissue thickness, and/or to promote tissue ingrowth when the adjuncts are stapled to tissue.

An exemplary stapling assembly can include a variety of features to facilitate application of a surgical staple, as described herein and illustrated in the drawings. However, a person skilled in the art will appreciate that the stapling assembly can include only some of these features and/or it can include a variety of other features known in the art. The stapling assemblies described herein are merely intended to represent certain exemplary examples. Moreover, while the adjuncts are described in connection with surgical staple cartridge assemblies, the adjuncts can be used in connection with staple reloads that are not cartridge based or any type of surgical instrument.

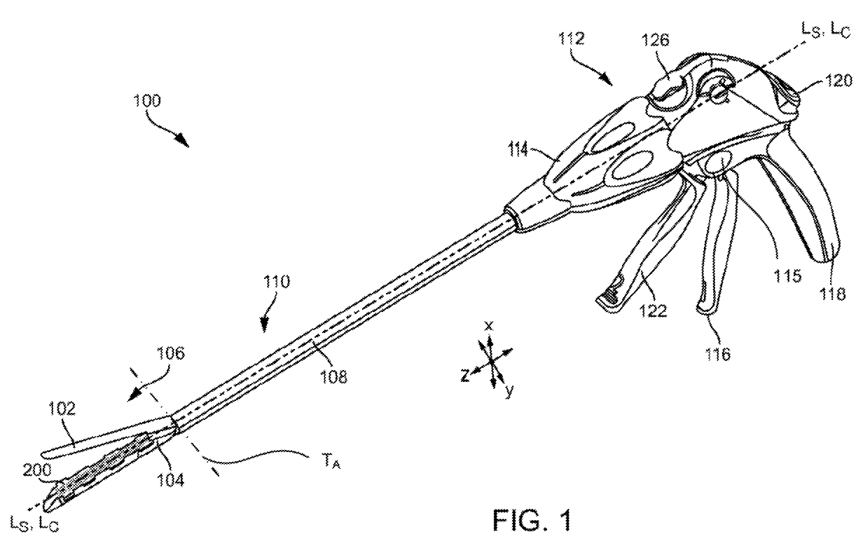

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the staple applying assembly 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108, forming an implement portion 110. When the end effector 106 is closed, or at least substantially closed (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (T_{A}) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be a powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

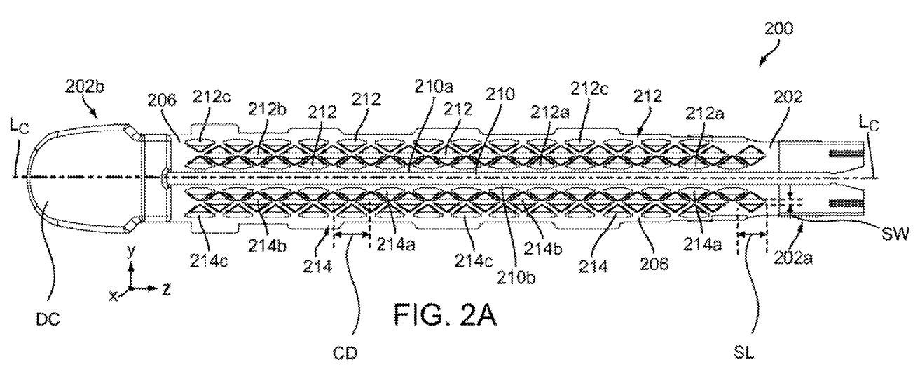

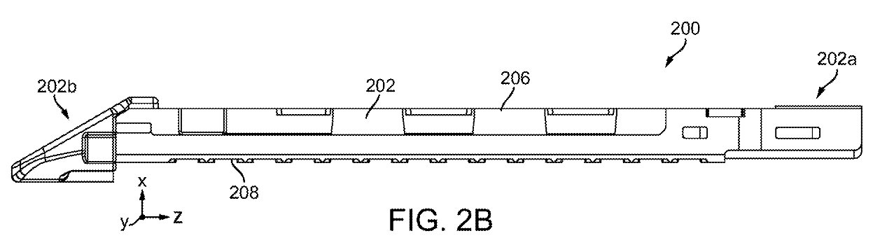

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B, has a proximal end 202a and a distal end 202b with a cartridge longitudinal axis (LC) extending therebetween. As a result, when the staple cartridge 200 is inserted into the elongate channel 104 (FIG. 1), the longitudinal axis (LC) is substantially or approximately parallel with the longitudinal axis (LS) of the elongate shaft 108. Further, the staple cartridge 200 includes a longitudinal slot 210 defined by two opposing walls 210a, 210b and configured to receive at least a portion of a firing member of a firing assembly, like firing assembly 400 in FIG. 4, as discussed further below. As shown, the longitudinal slot 210 extends from the proximal end 202a toward the distal end 202b of the staple cartridge 200. It is also contemplated herein that in other examples, the longitudinal slot 210 can be omitted.

The illustrated staple cartridge 200 includes staple cavities 212, 214 defined therein, in which each staple cavity 212, 214 is configured to removably house at least a portion of a staple (not shown). The number, shape, and position of the staple cavities can vary and can depend at least on the size and shape (e.g., mouth-like shape) of the staples to be removably disposed therein. In this illustrated example, the staple cavities are arranged in two sets of three longitudinal rows, in which the first set of staple cavities 212 is positioned on a first side of the longitudinal slot 210 and the second set of staple cavities 214 is positioned on a second side of the longitudinal slot 210. On each side of the longitudinal slot 210, and thus for each set of rows, a first longitudinal row of staple cavities 212a, 214a extends alongside the longitudinal slot 210, a second row of staple cavities 212b, 214b extends alongside the first row of staple cavities 212a, 214a, and a third row of staple cavities 212c, 214c extends alongside the second row of staple cavities 212b, 214b. Each row may be approximately parallel and the staple cavities that make up the rows may be approximately parallel in orientation with the longitudinal slot 210. As shown in FIGS. 2A, each staple cavity 212, 214 may include a maximum length SL of about 0.122 inches to about 0.124 inches and a maximum width SW of about 0.023 inches to about 0.027 inches. In addition, at least the centers of two adjacent cavities 212, 214 are spaced apart by about 0.158 inches.

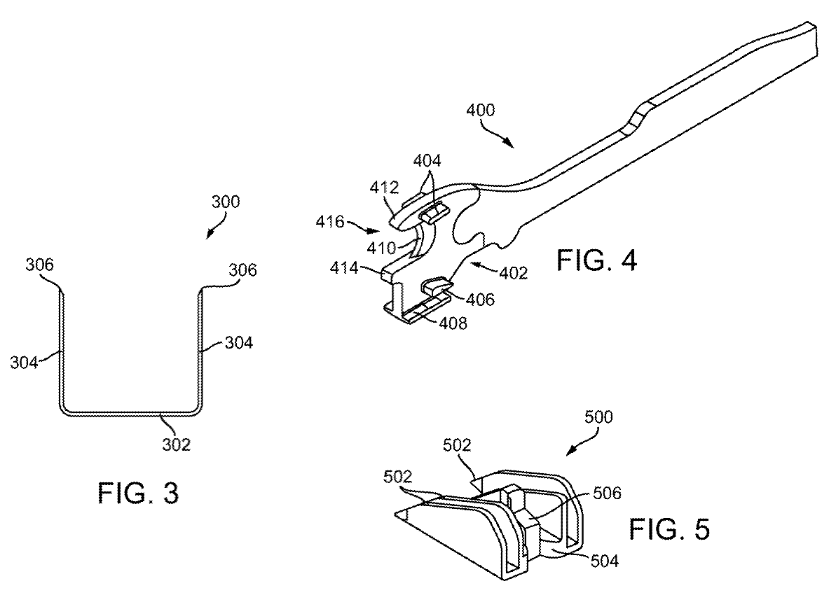

The staples releasably stored in the staple cavities 212, 214 can have a variety of configurations. An exemplary staple 300 (shown in FIG. 3) that can be releasably stored in each of the staple cavities 212, 214 in its unfired (pre-deployed, unformed) configuration. The illustrated staple 300 includes a crown (base) 302 and two legs 304 extending from each end of the crown 302. In this example, the crown 302 extends in a linear direction and the staple legs 304 have the same unformed height. Further, prior to the staples 300 being deployed, the staple crowns 302 can be supported by staple drivers that are positioned within the staple cartridge 200 and, concurrently, the staple legs 304 can be at least partially contained within the staple cavities 212, 214. Further, the staple legs 304 can extend beyond a top surface, like top surface 206, of the staple cartridge 200 when the staples 300 are in their unfired positions. In certain instances, as shown in FIGS. 3 and 8A, the tips 306 of the staple legs 304 can be pointed and sharp which can incise and penetrate tissue.

In use, staples 300 can be deformed from an unfired position into a fired position such that the staple legs 304 move through the staple cavities 212, 214, penetrate tissue positioned between the anvil 102 and the staple cartridge 200, and contact the anvil 102. As the staple legs 304 are deformed against the anvil 102, the legs 304 of each staple 300 can capture a portion of the tissue within each staple 300 and apply a compressive force to the tissue. Further, the legs 304 of each staple 300 can be deformed downwardly toward the crown 302 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed, unformed) configuration. In other examples, the staples can include at least two groups of staples each having a different unfired (pre-deployed, unformed) configuration, e.g., varying in height and/or shape, relative to one another, etc.

Referring back to FIGS. 2A-2B, the staple cartridge 200 extends from a top surface or deck surface 206 to a bottom surface 208, in which the top surface 206 is configured as a tissue-facing surface and the bottom surface 208 is configured as a channel-facing surface. As a result, when the staple cartridge 200 is inserted into the elongate channel 104, as shown in FIG. 1, the top surface 206 faces the anvil 102 and the bottom surface 208 (obstructed) faces the elongate channel 104.

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that affects staple formation by the end effector 106.

In use, the anvil 102 in FIG. 1 can be moved into a closed position by depressing the closure trigger in FIG. 1 to advance the E-beam 402 in FIG. 4. The anvil 102 can position tissue against at least the top surface 206 of the staple cartridge 200 in FIGS. 2A-2B. Once the anvil has been suitably positioned, the staples 300 in FIG. 3 disposed within the staple cartridge can be deployed.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be essentially fully absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

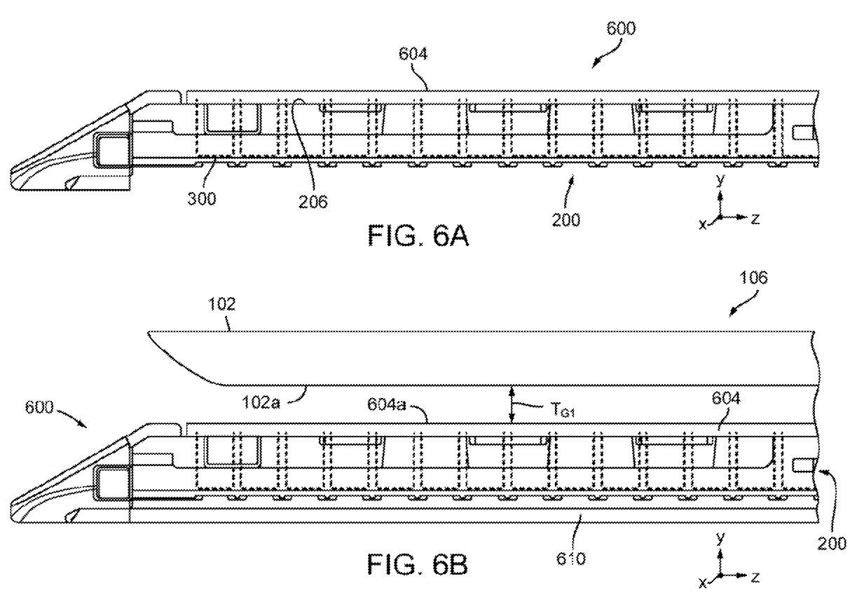

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300, that are configured to be deployed into tissue. The staples 300 can have any suitable unformed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Patent Publication No. 2016/0106427, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap T_{G1}created between the staple cartridge 602 and the anvil 612. More specifically, the tissue gap T_{G1} is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap T_{G1} is generally uniform (e.g., nominally identical within manufacturing tolerances) when no tissue is disposed therein. In other words, the tissue gap T_{G1} is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap T_{G1} can be varied.

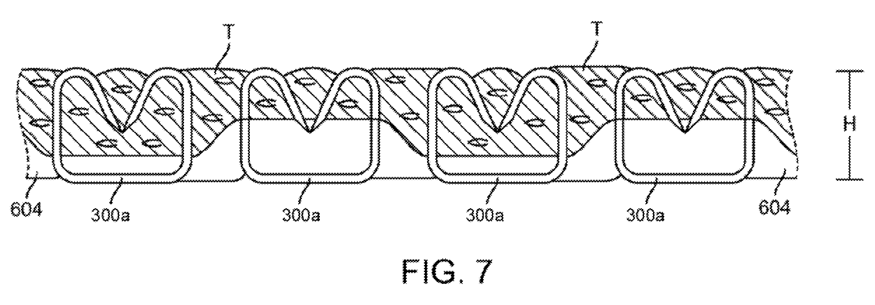

The adjunct 604 is compressible to permit the adjunct to compress to varying heights to thereby compensate for different tissue thickness that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7). That is, the adjunct 604 can have an undeformed state in which a maximum height of the adjunct 604 is greater than a maximum height of a fired staple (e.g., a staple that is in a formed configuration).

As shown in FIG. 7, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

The surgical adjunct 604 needs to have mechanical properties such that the material is sufficiently compressible during delivery to be inserted through a trocar, but must have increased strength to retain staples, sutures, and screws at the delivery site. In addition, the surgical adjunct 604 needs to be bioabsorbable such that after delivery, the adjunct 604 remains at the target site until the tissue can sufficiently maintain a hemostatic seal and/or the tissue is sufficiently healed. From a mechanical standpoint, the adjunct 604 provides an ideal amount of compression that is both strong enough to create and maintain the hemostatic seal and is not over-compressing or too tough to permit the firing of the staples upon delivery. A short-term absorption profile may be preferred to address hemostasis while a long-term absorption profile may address better tissue healing without leakages.

In various embodiments, the adjunct or bioabsorbable materials disclosed herein can be comprised of an absorbable polymer. In certain embodiments, an adjunct can be comprised of foam, film, fibrous woven, fibrous non-woven polyurethane, polyester, polycarbonate, polyorthoesters, polyanhydrides, polyesteramides, and/or polyoxaesters. In other embodiments, an adjunct can be a copolymer including, for example, PGA (polyglycolic acid), PGA/PCL (poly(glycolic acid-co-caprolactone)), PLA/PCL (poly(lactic acid-co-polycaprolactone)), PLLA/PCL, PGA/TMC (poly(glycolic acid-co-trimethylene carbonate)), PDS, PEPBO, and the like. In various embodiments, an adjunct can include an organic material such as, for example, carboxymethyl cellulose, sodium alginate, hyaluronic acid, and/or oxidized regenerated cellulose. In various embodiments, an adjunct has a durometer in the 3-7 Shore A (30-50 Shore OO) ranges with a maximum stiffness of 15 Shore A (65 Shore OO). In certain embodiments, an adjunct can undergo 40% compression under 3 lbf load, 60% compression under 6 lbf load, and/or 80% compression under 20 lbf load, for example. In certain embodiments, one or more gasses, such as air, nitrogen, carbon dioxide, and/or oxygen, for example, can be bubbled through and/or contained within the adjunct.

In some embodiments, the adjunct 604 can comprise a bioabsorbable material that includes a polymerizable compound that has a degradation profile. The material choice of the polymerizable compound can be a reaction product of a polyol and an isocyanate, such as polyurethane or a like polymer including, without limitation, polyether urethane, polyester urethane, polyester urea, polyester, polycarbonate, polyorthoester, polyanhydride, polyesteramide, polyphosphazenes, polyphosphoesters, polysaccharides, and/or polyoxaester. The polyurethane reaction product can have a degradation profile that is tailored for the target tissue site. In some embodiments, the adjunct 604 described can be a polymer with tunable absorption properties via an alternating copolymer, a random copolymer, a block copolymer, a multiblock copolymer architecture, a terpolymer, a graft copolymer (monomer backbone with pendant chains of a different monomer), a bulk homopolymer coated with a second homopolymer, and the like. The surgical adjunct 604 can be tuned for a particular purpose before, during, and after surgical procedures. In particular, the adjunct 604 described has selective crosslinking density control in vivo and can also compressibility when attached to the cartridge outside of the body.

A specific example of a stapling adjunct comprises a PU foam. The primary absorption mechanism of PU involves the oxidation of labile amine crosslinkers. During a foreign body response, macrophages release numerous enzymes and oxidants. The presence of metallic components can catalyze oxidation through a mechanism called metal ion oxidation. The catalyzation mechanism can occur through various pathways including direct oxidation of the metal before initiating any PU oxidation or degradation. This is of particular interest as oxidation can occur in the absence of a foreign body response and independent of the macrophage presence. Therefore, the presence of a metallic component in the adjunct can be used to degrade the PU via an anaerobic and autoxidation process.

The catalyzation rate can be tuned by adjusting the oxidation potential and the type of metal. While specific oxidation potential is dependent on the surrounding solution (e.g., ion balance), general trends can be understood based on the values of a metal’s oxidation potential.

The present disclosure describes a rapidly absorbing surgical adjunct for a shorter implant presence time in vivo while maintaining mechanical strength for a sufficiently long time for healing. The adjunct comprises a PU foam and a metallic component, wherein the metallic component accelerates the degradation of the foamin vivo. The present disclosure provides various advantages, including an increased absorption rate for the adjunct, a reduced sensitivity to foreign body response, and a spatially tunable absorption profile of the adjunct depending on the location of the metallic component.

A metallic component may be included in the adjunct physically, wherein there is no chemical bond between a metal and any part of the PU.

Provided herein is surgical adjunct 604, comprising a polyurethane (PU) foam and a metallic component, wherein the metallic component accelerates the degradation of the foam in vivo.

In some embodiments, the volumetric ratio of the metallic component to the total volume of the surgical adjunct 604 is less than about 10%. In some embodiments, the volumetric ratio of the metallic component to the total volume of the surgical adjunct 604 is about 0.01% to about 10%. In some embodiments, the volumetric ratio of the metallic component to the total volume of the surgical adjunct 604 is about 0.01% to about 0.1%, about 0.01% to about 1%, about 0.01% to about 2%, about 0.01% to about 5%, about 0.01% to about 8%, 0.1% to about 1%, about 0.1% to about 2%, about 0.1% to about 5%, about 0.1% to about 8%, about 1% to about 2%, about 1% to about 5%, about 1% to about 8%, about 2% to about 5%, about 2% to about 8%, or about 5% to about 8%.

In some embodiments, the volumetric ratio of the polyurethane foam to the total volume of the surgical adjunct 604 in a range of about 0.125 to about 0.325, and wherein a glass transition temperature of the surgical adjunct 604 is about 0°C to about 40°C.

In some examples, the adjunct 604 includes a film disposed on one or more surfaces of the polyurethane foam. In some embodiments, the film is a moisture barrier film including aliphatic polyesters such as polylactic acid (PLA), polyglycolic acid (PGA), polycaprolactone (PCL), polydioxanone (PDO), poly(trimethylene carbonate) (PTMC), or copolymer combinations. In some embodiment, the adjunct 604 includes a polydioxanone (PDO) film disposed on one or more surfaces of the polyurethane foam. In some examples, the PDO film is adhered to at least a bottom or crown side of the adjunct 604. In some examples, the PDO film has a thickness of about 20 µm to about 100 µm, such as about 40 µm.

In some embodiments, the metallic component comprises one or more metals, one or more metallic compounds, or any combination thereof.

In some embodiments, the metallic component comprises one or more metallic elements selected from alkali metals, alkaline-earth metals, or transition metals. In some embodiments, the metal comprises one or more elements selected from the group consisting of Ag, Al, Au, Ca, Co, Cr, Cu, Fe, K, Li, Mg, Mo, Mn, Na, Ni, Pd, Pt, Rh, Ru, Sn, Ti, V, W, Zn, and Zr.

In some embodiments, the metallic compound comprises one or more metal elements selected from the group consisting of Ag, Al, Au, Co, Cr, Cu, Fe, Ni, Mg, Mo, Mn, Pd, Pt, Rh, Ru, Sn, Ti, V, W, Zn and Zr and one or more elements selected from the group consisting of B, C, H, O, P and S. In some embodiments, the metallic compound is a metallic oxide. In some embodiments, the metallic oxide is an oxide of Ca, Fe, Li, Mg, Mn, Na, K, Sn, or Ti.

In some embodiments, the metallic compound is a metallic salt. In some embodiments, the metallic salt comprises one or more cations selected from the group consisting of Li, Na, K, Ca, Ag, Al, Co, Cr, Cu, Fe, Ni, Mg, Mo, Mn, Pd, Rh, Ru, Sn, Ti, V, W, Zn and Zr. In some embodiments, the metallic salt comprises one or more anions selected from the group consisting of a halogen anion, acetylacetonate anion, acetate, butyrate, caprylate, carbonate, citrate, lactate, malate, oxalate, tartarate, succinate, propionate, ClO⁻, SO₄^{2−}, PO₄^{3−}, and NO₃⁻.

In some embodiments, the metallic salt is an inorganic salt. In some embodiments, the inorganic salt comprises one or more anions selected from the group consisting of a halogen anion, acetylacetonate anion, acetate, butyrate, caprylate, carbonate, citrate, lactate, malate, oxalate, tartarate, succinate, propionate, ClO⁻, SO₄^{2−}, PO₄^{3−}, and NO₃⁻.

In some embodiments, the halogen ions are chloride, bromide, or fluoride ions.

In some embodiments, the metallic salt is an organic salt. In some embodiments, the organic salt comprises trifluoroacetate or maleate.

In some embodiments, the metallic salt comprises a sulfonate salt comprising methanesulfonate, ethanesulfonate, benzenesulfonate, p-toluenesulfonate, camphor sulfonate, or naphthalenesulfonate.

In some embodiments, the metallic salt comprises an amino acid salt comprising arginate, alaninate, asparginate, or glutamate.

In some embodiments, the metallic salt comprises carbohydrate salts such as gluconate and galacturonate.

In some embodiments, the metallic component is distributed in at least a portion of the polyurethane foam. In some embodiments, the metallic component is distributed on at least a portion of a surface of the polyurethane foam. The inclusion of the metallic component at the surface of the PU foam promotes an initial absorption acceleration that could minimize the amount of metal added to the tissue system. In the event that the metallic component is not removed from the implant area, the inclusion of the metallic component is preferred on the surface considering the surface-eroding nature of the oxidative mechanism. The inclusion of metallic components in the bulk of adjuncts may be useful to increase the overall absorption rate.

In some embodiments, the surgical adjunct 604 further comprises a polydioxanone (PDO) film disposed on one or more surfaces of the PDO foam, wherein the metal component is dispersed in at least a portion of PDO film or the PDO foam.

In some embodiments, a PDO film is disposed on one or more surfaces of the polyurethane foam. In some embodiments, a PDO film is disposed on one or more surfaces of the adjunct. In some embodiments, the metallic component is dispersed in at least a portion of PDO film. In some embodiments, the metallic component dispersed in at least a portion of the surface of the PDO film.

In some embodiments, the surgical adjunct 604 comprises a first portion that comprises a foam comprised of PU and a second portion that does not comprise a foam comprised of PU. In some embodiments, the first portion further comprises the metallic component. In some embodiments, the metallic component is dispersed in the first portion. In some embodiments, the metallic component is disposed on one or more surfaces of the portion of the first portion. In some embodiments, the first portion does not comprise the metallic component and the second portion comprises the metallic component. In some embodiments, the metallic component is dispersed in the second portion. In some embodiments, the metallic component is disposed on one or more surfaces of the portion of the second portion.

In some embodiments, one component of the end effector 106 comprises the metallic component and the surgical adjunct 604 does not comprise the metallic component. As a non-limiting example, the anvil 102 may comprise the metallic component, and contact between the anvil 102 and the metallic component can transfer the metallic component to the surgical adjunct 604. In some embodiments, one component of the end effector 106 comprises the metallic component and the surgical adjunct 604 also comprises the metallic component.

The inclusion of metal into the adjunct may be achieved during the PU foaming process (i.e., additive to a feed component) or as a secondary process after foaming has been completed. Given the potential extraction process required to minimize leachable components, the inclusion of a metal component should minimize any influence on the backbone and macroscopic PU foam structure. The metal can be included in an extraction solution such that during the PU swelling process, the metal may be entrapped within the polymer network.

A method is provided herein for preparing the surgical adjunct 604, including a step of adding the metallic component during a foaming process of the polyurethane; or depositing the metallic component onto at least a portion of a surface of the polyurethane foam by spray coating, dip coating, electrospray, pad printing, screen printing, material jetting, or direct deposition of the metallic component onto the surface.

In some embodiments, depositing the metallic component onto at least a portion of a surface of the polyurethane foam comprises:

coating at least a deck surface of a staple cartridge with the metallic component; and

attaching the polyurethane foam to the deck surface, thereby transferring at least a portion of the metallic component to the polyurethane foam.

In some embodiments, coating at least the deck surface of the staple cartridge comprises spray coating, dip coating, electrospray, pad printing, screen printing, material jetting, or direct deposition of the metallic component onto the staple cartridge.

Certain technologies (e.g., pad printing, screen printing, material jetting, direct deposition, etc.) have the advantage of placing the metallic component spatially across the stapling adjunct. This can be used to allow selective absorption rates within the adjunct.

A metallic component may be included in the adjunct chemically, wherein there is a chemical bond between a metal and a part of the PU. The chemical bond may be an ionic bond or a covalent bond. The inclusion of metal is achieved during the polymerization process. The attachment of the metal to the PU backbone or sidechain retains the metal locally for a longer duration (relative to the situation there is no chemical bonding between the metal and any part of the PU) to enable catalytic activity during the absorption process. The inclusion of the metal may be achieved along the PU backbone (i.e., inclusion via specific oligomer or monomer species) or as polymer brushes attached to the surface. The features for surface inclusion of the metal described above would apply to polymer brushes.

Provided herein is a surgical adjunct 604, having a metal-containing polyurethane foam.

In some embodiments, the one or more metallic elements are incorporated into a backbone or a sidechain of polyurethane, and wherein the metallic element accelerates the degradation of the foamin vivo.

In some embodiments, the metal-containing polyurethane is an organometallic compound.

In some embodiments, the metal-containing polyurethane is a metal-organic framework (MOF). In some embodiments, the MOF contains metal ions or clusters coordinated to organic molecules comprising PU.

In some embodiments, the metal-containing polyurethane is a reaction product of a polyol and an isocyanate, wherein the polyol, the isocyanate, or both comprise the one or more metallic elements.

In some embodiments, the metal-containing polyurethane is a reaction product of a polyol and an isocyanate, wherein the polyol comprises the one or more metallic elements.

In some embodiments, the metal-containing polyurethane is a reaction product of a polyol and an isocyanate, wherein the isocyanate comprises the one or more metallic elements.

In some embodiments, the metal-containing polyurethane is a reaction product of a polyol and an isocyanate, wherein both of the polyol and the isocyanate comprise the one or more metallic elements.

In some embodiments, the metallic element is selected from the group consisting of Ag, As, Au, Bi, Ca, Cd, Co, Cr, Cu, Eu, Fe, Ga, Gd, Ge, Hg, Ho, Ir, Mg, Mn, Ni, Os, Pb, Pt, Re, Ru, Sb, Se, Sn, Tc, Te, V, Y, Zn, and Zr.

In some embodiments, the one or more metallic elements are incorporated into a backbone or a sidechain of polyurethane.

In some embodiments, the sidechain includes a metallocene, a metal carbonyl group, or a ligated metallic group.

In some embodiments, the sidechain has a metallocene. In some embodiments, the metallocene is ferrocene, cobaltocene, ruthenocene, osmocene, or any combination thereof.

In some embodiments, the side chain comprises one or more polymeric metal chelates. In some embodiments, the one or more polymeric metal chelates comprises N,N-, N,О-, N,S-, О,О-, O,S-, S,S-, Р,Р-chelates, or any combination thereof.

In some embodiments, the side chain has ferrocene, cobaltocene, ruthenocene, osmocene, one or more polymeric metal chelates comprising N,N-, N,О-, N,S-, О,О-, O,S-, S,S-, or Р,Р-chelates.

In some embodiments, the O,O-chelate is derived from an acetoacetoxy.

In some embodiments, the side chain includes a derivative of porphyrin or a norbornene.

In some embodiments, the metallic element is incorporated into the backbone of polyurethane.

In some embodiments, the metal-containing polyurethane is a reaction product of a diisocyanate and a diol.

In some embodiments, the metal-containing polyurethane is a reaction product of a diisocyanate and a diol, wherein the diisocyanate, the diol, or both contain one or more metallic elements.

In some embodiments, the metal-containing polyurethane is a reaction product of a diisocyanate and a diol, wherein the diisocyanate contains one or more metallic elements.

In some embodiments, the metal-containing polyurethane is a reaction product of a diisocyanate and a diol, wherein the diol contains one or more metallic elements.

In some embodiments, the metal-containing polyurethane is a reaction product of a diisocyanate and a diol, wherein both of the diisocyanate and the diol contain one or more metallic elements.

In some embodiments, the diol is an ionic diol containing ionic linkages between one or more metal cations and one or more carboxylate anions.

In some embodiments, the ionic diol is a divalent metal ionic salt.

In some embodiments, the ionic diol comprises a group that is

mono(hydroxyethyl)phthalate, mono(hydroxybutyl)phthalate,

mono(hydroxyethoxyethyl)phthalate, mono(hydroxypentyl)phthalate, or

mono(hydroxybutyl)hexolate.

In some embodiments, the diisocyanate is hexamethylene diisocyanate (HMDI), tolylene 2,4-diisocyanate (TDI), or 4,4’ -diphenylmethane diisocyanate (MDI).

In some embodiments, the PU foam comprises a first PU portion comprising metal-containing PU containing a metal in the backbone or sidechain of PU and a second PU portion not containing a metal in the backbone or sidechain of PU.

In some embodiments, the metal-containing PU is distributed in at least a portion of the PU foam. In some embodiments, the metal-containing PU is distributed on at least a portion of the surface of the PU foam.

When there is no chemical bond between metal and PU, certain metals may be removed from the PU foam through biological processes quickly. The effective exposure time of the metal to the PU may be reduced, thus limiting the catalyzation effect. The metal-containing PU has the advantages of localization and retention of catalyst near the PU molecule. Thus, a reduced amount of metal can be used for an equivalent catalyzation effect.

A metallic component may not be included in the surgical adjunct 604. The metallic component may be included in other components of the surgical stapler or in surgical staples. At least a portion of the metallic component may be transferrable to a portion of the surgical adjunct 604 comprising a PU foam, such that the portion of the metallic component accelerates the degradation of the foam in vivo.

Provided herein is a system comprising:

a staple cartridge 200 comprising a deck surface 206 with a plurality of staple cavities 212,

214, each staple cavity of the plurality of staple cavities 212, 214 comprising a staple 300;

a surgical adjunct 604, coupled to the deck surface 206 and comprising a PU foam; and

a metallic component configured to accelerate the degradation of the foam in vivo.

In some embodiments, one component of the staple cartridge 200 comprises the metallic component, and the metallic component is transferrable to the PU foam in response to the PU foam contacting the component of the staple cartridge 200 comprising the metallic component.

In some embodiments, one component of the staple cartridge 200 comprises the metallic component and the surgical adjunct 604 does not comprise the metallic component. In some embodiments, one component of the staple cartridge 200 comprises the metallic component and the surgical adjunct 604 also comprises the metallic component.

In some embodiments, the component of the staple cartridge 200 comprising the metallic comprises at least one of the staple 300, the deck surface 206, a knife, or a sled 500.

As will be appreciated by one skilled in the art, the embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

In some examples, disclosed devices (e.g., end effector, surgical adjunct, and/or staple cartridges) and methods involving one or more disclosed devices may involve one or more of the following clauses:

Clause 1. A surgical adjunct 604, comprising a polyurethane (PU) foam and a metallic component, wherein the metallic component accelerates the degradation of the foam in vivo.

Clause 2. The surgical adjunct 604 of clause1, comprising a volumetric ratio of the metallic component to the total volume of the surgical adjunct 604 less than about 10%.

Clause 3. The surgical adjunct 604 of clause 1 or clause 2, comprising a volumetric ratio of the polyurethane foam to the total volume of the surgical adjunct 604 in a range of about 0.125 to about 0.325, and wherein a glass transition temperature of the surgical adjunct 604 is about 0°C to about 40°C.

Clause 4. The surgical adjunct 604 of any one of clauses1-3, wherein the metallic component comprises one or more metals, one or more metallic compounds, or any combination thereof.

Clause 5. The surgical adjunct 604 of any one of clauses 1-4, wherein the metallic component comprises one or more metallic elements selected from alkali metals, alkaline-earth metals, or transition metals, optionally wherein the metal comprises one or more elements selected from the group consisting of Ag, Al, Au, Ca, Co, Cr, Cu, Fe, K, Li, Mg, Mo, Mn, Na, Ni, Pd, Pt, Rh, Ru, Sn, Ti, V, W, Zn, and Zr.

Clause 6. The surgical adjunct 604 of any one of clauses 1-4, wherein the metallic compound comprises one or more metal elements selected from the group consisting of Ag, Al, Au, Co, Cr, Cu, Fe, Ni, Mg, Mo, Mn, Pd, Pt, Rh, Ru, Sn, Ti, V, W, Zn and Zr and one or more elements selected from the group consisting of B, C, H, O, P and S, optionally wherein the metallic compound is a metallic oxide, further optionally an oxide of Ca, Fe, Li, Mg, Mn, Na, K, Sn, or Ti.

Clause7. The surgical adjunct 604 of any one of clauses 1-4, wherein the metallic compound is a metallic salt, optionally comprising one or more cations selected from the group consisting of Li, Na, K, Ca, Ag, Al, Co, Cr, Cu, Fe, Ni, Mg, Mo, Mn, Pd, Rh, Ru, Sn, Ti, V, W, Zn and Zr, further optionally comprising one or more anions selected from the group consisting of a halogen anion, acetylacetonate anion, acetate, butyrate, caprylate, carbonate, citrate, lactate, malate, oxalate, tartarate, succinate, propionate, ClO−, SO42−, PO43−, and NO3−.

Clause 8. The surgical adjunct 604 of any one of clauses 1-7, wherein the metallic component is distributed in at least a portion of the polyurethane foam, optionally on at least a portion of a surface of the polyurethane foam.

Clause 9. The surgical adjunct 604 of any one of clauses 1-8, further comprising a polydioxanone (PDO) film disposed on one or more surfaces of the polyurethane foam, wherein the metallic component is dispersed in at least a portion of the polydioxanone film or the polyurethane foam.

Clause 10. A method of preparing the surgical adjunct 604 of any one of clauses1-8, comprising adding the metallic component during a foaming process of the polyurethane; or depositing the metallic component onto at least a portion of a surface of the polyurethane foam by spray coating, dip coating, electrospray, pad printing, screen printing, material jetting, or direct deposition.

Clause 11. The method of clause 10, wherein depositing the metallic component onto at least a portion of a surface of the polyurethane foam comprises spray coating, dip coating, electrospray, pad printing, screen printing, material jetting, or direct deposition of the metallic component onto the surface.

Clause 12. The method of clause 10, wherein depositing the metallic component onto at least a portion of a surface of the polyurethane foam comprises:

coating at least a deck surface of a staple cartridge with the metallic component; and

attaching the polyurethane foam to the deck surface, thereby transferring at least a portion of the metallic component to the polyurethane foam.

Clause 13. The method of clause 12, wherein coating at least the deck surface of the staple cartridge comprises spray coating, dip coating, electrospray, pad printing, screen printing, material jetting, or direct deposition of the metallic component onto the staple cartridge.

Clause 14. A surgical adjunct 604, comprising a metal-containing polyurethane foam, wherein one or more metallic elements are incorporated into a backbone or a sidechain of polyurethane, and wherein the metallic element accelerates the degradation of the foam in vivo.

Clause 15. The surgical adjunct 604 of clause 14, wherein the metal-containing polyurethane is a reaction product of a polyol and an isocyanate, wherein the polyol, the isocyanate, or both comprise the one or more metallic elements.

Clause 16. The surgical adjunct 604 of clause 14 or clause 15, wherein the metallic element is selected from the group consisting of Ag, As, Au, Bi, Ca, Cd, Co, Cr, Cu, Eu, Fe, Ga, Gd, Ge, Hg, Ho, Ir, Mg, Mn, Ni, Os, Pb, Pt, Re, Ru, Sb, Se, Sn, Tc, Te, V, Y, Zn, and Zr.

Clause 17. The surgical adjunct 604 of any one of clauses 14-16, wherein the sidechain comprises a metallocene, a metal carbonyl group, or a ligated metallic group.

Clause 18. The surgical adjunct 604 of any one of clauses 14-17, wherein the side chain comprises ferrocene, cobaltocene, ruthenocene, osmocene, one or more polymeric metal chelates comprising N,N-, N,О-, N,S-, О,О-, O,S-, S,S-, or Р,Р-chelates, optionally O,O-chelates derived from an acetoacetoxy, or a derivative of porphyrin or a norbornene.

Clause 19. The surgical adjunct 604 of any one of clauses 14-16, wherein when the metallic element is incorporated into the backbone of polyurethane, the metal-containing polyurethane is a reaction product of a diisocyanate; and a diol containing one or more metallic elements.

Clause 20. The surgical adjunct 604 of clause 19, wherein the diol is an ionic diol containing ionic linkages between one or more metal cations and one or more carboxylate anions, optionally the ionic diol is a divalent metal ionic salt comprising a group that is

mono(hydroxyethyl)phthalate, mono(hydroxybutyl)phthalate,

mono(hydroxyethoxyethyl)phthalate, mono(hydroxypentyl)phthalate, or

mono(hydroxybutyl)hexolate, further optionally wherein the diisocyanate is hexamethylene diisocyanate (HMDI), tolylene 2,4-diisocyanate (TDI), or 4,4’ -diphenylmethane diisocyanate (MDI).

Clause 21. A system comprising:

a staple cartridge 200 comprising a deck surface 206 with a plurality of staple cavities 212, 214, each staple cavity of the plurality of staple cavities 212, 214 comprising a staple 300;

a surgical adjunct 604, coupled to the deck surface 206 and comprising a polyurethane (PU) foam; and

a metallic component configured to accelerate the degradation of the foam in vivo.

Clause 22. The system of clause 21, one component of the staple cartridge 200 comprises the metallic component, and the metallic component is transferrable to the PU foam in response to the PU foam contacting the component of the staple cartridge 200 comprising the metallic component.

Clause 23. The system of clause 22, wherein:

the first component comprises at least one of the staple 300, the deck surface 206, a knife, or a sled 500; or

the second component comprises an anvil 102 of the end effector 106.

The present application is not to be limited in scope by the specific embodiments described herein. Indeed, various modifications of the embodiments in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. A surgical adjunct 604, comprising a polyurethane (PU) foam and a metallic component, wherein the metallic component accelerates the degradation of the foam in vivo.

2. The surgical adjunct 604 of claim 1, comprising a volumetric ratio of the metallic component to the total volume of the surgical adjunct 604 less than about 10%.

3. The surgical adjunct 604 of claim 2, comprising a volumetric ratio of the polyurethane foam to the total volume of the surgical adjunct 604 in a range of about 0.125 to about 0.325, and wherein a glass transition temperature of the surgical adjunct 604 is about 0°C to about 40°C.

4. The surgical adjunct 604 of claim 1, wherein the metallic component comprises one or more metals, one or more metallic compounds, or any combination thereof.

5. The surgical adjunct 604 of claim 1, wherein the metallic component comprises one or more metallic elements selected from alkali metals, alkaline-earth metals, or transition metals, optionally wherein the metal comprises one or more elements selected from the group consisting of Ag, Al, Au, Ca, Co, Cr, Cu, Fe, K, Li, Mg, Mo, Mn, Na, Ni, Pd, Pt, Rh, Ru, Sn, Ti, V, W, Zn, and Zr.

6. The surgical adjunct 604 of claim 1, wherein the metallic compound comprises one or more metal elements selected from the group consisting of Ag, Al, Au, Co, Cr, Cu, Fe, Ni, Mg, Mo, Mn, Pd, Pt, Rh, Ru, Sn, Ti, V, W, Zn and Zr and one or more elements selected from the group consisting of B, C, H, O, P and S, optionally wherein the metallic compound is a metallic oxide, further optionally an oxide of Ca, Fe, Li, Mg, Mn, Na, K, Sn, or Ti.

7. The surgical adjunct 604 of claim 1, wherein the metallic compound is a metallic salt, optionally comprising one or more cations selected from the group consisting of Li, Na, K, Ca, Ag, Al, Co, Cr, Cu, Fe, Ni, Mg, Mo, Mn, Pd, Rh, Ru, Sn, Ti, V, W, Zn and Zr, further optionally comprising one or more anions selected from the group consisting of a halogen anion, acetylacetonate anion, acetate, butyrate, caprylate, carbonate, citrate, lactate, malate, oxalate, tartarate, succinate, propionate, ClO⁻ , SO₄^{2−} , PO₄^{3−} , and NO₃⁻.

8. The surgical adjunct 604 of claim 1, wherein the metallic component is distributed in at least a portion of the polyurethane foam.

9. The surgical adjunct 604 of claim 1, further comprising a polydioxanone (PDO) film disposed on one or more surfaces of the polyurethane foam, wherein the metallic component is dispersed in at least a portion of the polydioxanone film or the polyurethane foam.

10. A method of preparing the surgical adjunct 604 of claim 1, comprising:
adding the metallic component during a foaming process of the polyurethane; or
depositing the metallic component onto at least a portion of a surface of the polyurethane foam.

11. The method of claim 10, wherein depositing the metallic component onto at least a portion of a surface of the polyurethane foam comprises spray coating, dip coating, electrospray, pad printing, screen printing, material jetting, or direct deposition of the metallic component onto the surface.

12. The method of claim 10, wherein depositing the metallic component onto at least a portion of a surface of the polyurethane foam comprises:
coating at least a deck surface of a staple cartridge with the metallic component; and
attaching the polyurethane foam to the deck surface, thereby transferring at least a portion of the metallic component to the polyurethane foam.

13. A surgical adjunct 604, comprising a metal-containing polyurethane foam, wherein one or more metallic elements are incorporated into a backbone or a sidechain of polyurethane, and wherein the metallic element accelerates the degradation of the foam in vivo.

14. The surgical adjunct 604 of claim 13, wherein the metal-containing polyurethane is a reaction product of a polyol and an isocyanate, wherein the polyol, the isocyanate, or both comprise the one or more metallic elements.

15. The surgical adjunct 604 of claim 14, wherein the metallic element is selected from the group consisting of Ag, As, Au, Bi, Ca, Cd, Co, Cr, Cu, Eu, Fe, Ga, Gd, Ge, Hg, Ho, Ir, Mg, Mn, Ni, Os, Pb, Pt, Re, Ru, Sb, Se, Sn, Tc, Te, V, Y, Zn, and Zr.

16. The surgical adjunct 604 of claim 13, wherein the sidechain comprises a metallocene, a metal carbonyl group, or a ligated metallic group.

17. The surgical adjunct 604 of claim 16, wherein the side chain comprises ferrocene, cobaltocene, ruthenocene, osmocene, one or more polymeric metal chelates comprising N,N-, N,О-, N,S-, О,О-, O,S-, S,S-, or Р,Р-chelates.

18. The surgical adjunct 604 of claim 13, wherein when the metallic element is incorporated into the backbone of polyurethane, the metal-containing polyurethane is a reaction product of a diisocyanate; and a diol containing one or more metallic elements.

19. The surgical adjunct 604 of claim 18, wherein the diol is an ionic diol containing ionic linkages between one or more metal cations and one or more carboxylate anions, optionally the ionic diol is a divalent metal ionic salt comprising a group that is mono(hydroxyethyl)phthalate, mono(hydroxybutyl)phthalate, mono(hydroxyethoxyethyl)phthalate, mono(hydroxypentyl)phthalate, or mono(hydroxybutyl)hexolate, further optionally wherein the diisocyanate is hexamethylene diisocyanate (HMDI), tolylene 2,4-diisocyanate (TDI), or 4,4’ -diphenylmethane diisocyanate (MDI).

20. A system comprising:
a staple cartridge 200 comprising a deck surface 206 with a plurality of staple cavities 212, 214, each staple cavity of the plurality of staple cavities 212, 214 comprising a staple 300;
a surgical adjunct 604, coupled to the deck surface 206 and comprising a polyurethane (PU) foam; and
a metallic component configured to accelerate the degradation of the foam in vivo, wherein at least one of (i) a first component of the staple cartridge 200 or (ii) a second component of an end effector 106 comprises the metallic component, and the metallic component is transferrable to the PU foam in response to the PU foam contacting the first component or the second component.
